(19) United States
(12) Patent Application Publication
(10) Pub. No.: US 2024/0009366 A1
(43) Pub. Date: Jan. 11, 2024

(54) System and Method for Pressure Sensor Based Gas Bubble Detection for a Drug Delivery Device

(71) Applicant: Becton, Dickinson and Company, Franklin Lakes, NJ (US)

(72) Inventors: Steve Beguin, Rathdrum (IE); Sean McGrath, Artane (IE)

(21) Appl. No.: 17/859,627

(22) Filed: Jul. 7, 2022

(51) Int. Cl. A61M 1/36

(52) U.S. Cl. CPC A61M 1/3626; A61M 2205/502; A61M 2205/3355

(57) ABSTRACT

A drug delivery device may include a pressure sensor, a microprocessor, a fluid pathway including a reservoir, a pump downstream of the reservoir, and/or a fluid line downstream of the pump. The reservoir may be configured to receive a fluid, and the pump may be configured to deliver the fluid from the reservoir to the fluid line. The pressure sensor may be configured to measure a pressure in the fluid pathway downstream of the pump. The microcontroller may be programmed and/or configured to: receive, from the pressure sensor, the pressure measured in the fluid pathway downstream of the pump as the fluid is delivered to the fluid line; determine, based on the pressure measured in the fluid pathway, whether the fluid delivered to the fluid line includes a gas bubble; and control an output device to provide an indication associated with the determination a gas bubble.

### BACKGROUND

### Field

The present disclosure relates to a device and a method for pressure sensor based gas bubble detection for a drug delivery device.

### Description of Related Art

Wearable medical devices, such as automatic injectors, have a benefit of providing therapy to a patient at a location remote from a clinical facility and/or while being worn discretely under the patient's clothing. A wearable medical device can be applied to the patient's skin and configured to automatically deliver a dose of a pharmaceutical composition within a predetermined time period after applying the wearable medical device to the patient's skin, such as after a 27 hour delay. After the device delivers the pharmaceutical composition to the patient, the patient may subsequently remove and dispose of the device.

In the context of fluid injection or infusion, some drugs may be prescribed to be administered with a tightly controlled dose regimen (e.g., a precise doses may be prescribed to be delivered with controlled timing, etc.). Injection devices typically allow for a controlled flowrate and dosing regimen based on volumetric dosing, where a system imposes a known volumetric displacement of the fluid being administered. Exemplary systems allowing for volumetric-based control of dosing are syringe pumps, oscillo-rotative pumps, systems with moving piston, peristaltic pumps, and membrane and diaphragm pumps.

A subset of these pumps work based on a “dosing chamber element” that is cyclically filled and emptied. A particularity of these systems is that the fluidic path upstream the pump may not be in direct communication with the path downstream the pump, allowing for different pressures to be established in these respective fluid paths.

The presence of gas or air bubbles in fluid is a ubiquitous challenge in the field of precise dosing, as these gas bubbles impair the control of drug volume being administered by occupying some of the volume transiting through the fluid path, which may lead to under-dosing and/or may negatively impact a therapeutic treatment being administered. Solutions to air bubbles exist in the form of bubble traps and optical or ultrasonic bubble detection systems. However, these systems impose to add components in the fluid path or use sensors that may be expensive and/or consume significant power, which may be especially of concern for wearable devices.

### SUMMARY

Accordingly, provided are improved systems, devices, products, apparatus, and/or methods for gas bubble detection for a drug delivery device.

According to some non-limiting embodiments or aspects, provided is a drug delivery device, including: a fluid pathway including a reservoir, a pump downstream of the reservoir, and a fluid line downstream of the pump, wherein the reservoir is configured to receive a fluid, and wherein the pump is configured to deliver the fluid from the reservoir to the fluid line; a pressure sensor configured to measure a pressure in the fluid pathway downstream of the pump; and a microcontroller programmed and/or configured to: receive, from the pressure sensor, the pressure measured in the fluid pathway downstream of the pump as the fluid is delivered to the fluid line; determine, based on the pressure measured in the fluid pathway downstream of the pump as the fluid is delivered to the fluid line, whether the fluid delivered to the fluid line includes a gas bubble; and control an output device to provide an indication associated with the determination of whether the fluid delivered to the fluid line includes a gas bubble.

In some non-limiting embodiments or aspects, the pump includes a dosing chamber, wherein the pump is configured to cyclically (i) pump the reservoir with the dosing chamber in fluid communication with the reservoir and not in fluid communication with the fluid line and (ii) fluidically connect the dosing chamber with the fluid line, and wherein the fluid is delivered to the fluid line when the dosing chamber is fluidically connected with the fluid line.

In some non-limiting embodiments or aspects, the microcontroller is further programmed and/or configured to: receive, from the pressure sensor, a pressure measured in the fluid pathway downstream of the pump before fluidically connecting the dosing chamber with the fluid line; and determine, based on the pressure measured in the fluid pathway downstream of the pump before fluidically connecting the dosing chamber with the fluid line, a baseline pressure, wherein the microcontroller is programmed and/or configured to determine whether the fluid delivered to the fluid line includes a gas bubble based on the pressure measured in the fluid pathway downstream of the pump as the fluid is delivered to the fluid line and the baseline pressure.

In some non-limiting embodiments or aspects, the microcontroller is programmed and/or configured to determine whether the fluid delivered to the fluid line includes a gas bubble by: comparing the pressure measured in the fluid pathway downstream of the pump as the fluid is delivered to the fluid line to a threshold pressure; and in response to the pressure measured in the fluid pathway downstream of the pump as the fluid is delivered to the fluid line satisfying the threshold pressure, determining that the fluid delivered to the fluid line includes a gas bubble.

In some non-limiting embodiments or aspects, the microcontroller is programmed and/or configured to determine whether the fluid delivered to the fluid line includes a gas bubble by: determining, based on the pressure measured in the fluid pathway downstream of the pump as the fluid is delivered to the fluid line, a rate of change associated with the pressure measured in the fluid pathway downstream of the pump as the fluid is delivered to the fluid line; comparing the rate of change associated with the pressure measured in the fluid pathway downstream of the pump as the fluid is delivered to the fluid line to a threshold rate of change; and in response to the rate of change associated with the pressure measured in the fluid pathway downstream of the pump as the fluid is delivered to the fluid line satisfying the threshold rate of change, determine that the fluid delivered to the fluid line includes a gas bubble.

In some non-limiting embodiments or aspects, the microcontroller is further programmed and/or configured to: receive a pressure associated with the fluid pathway upstream of the pump; and determine, based on the pressure associated with the fluid pathway upstream of the pump, the pressure measured in the fluid pathway downstream of the pump as the fluid is delivered to the fluid line, and dimensions of the fluid pathway, at least one of a volume of the gas bubble included in the fluid delivered to the fluid line, a volume of the fluid delivered to the fluid line, or any combination thereof.

In some non-limiting embodiments or aspects, the microcontroller is programmed and/or configured to determine whether the fluid delivered to the fluid line includes a gas bubble by: determining, based on the pressure measured in the fluid pathway downstream of the pump as the fluid is delivered to the fluid line, a variation in the pressure over a period of time; comparing the variation in the pressure over the period of time to a threshold variation; and in response to the variation in the pressure over the period of time satisfying the threshold variation, determining that the fluid delivered to the fluid line during the period of time includes a gas bubble.

In some non-limiting embodiments or aspects, the microcontroller is further programmed and/or configured to: determine, based on the variation in the pressure over the period of time, at least one of a volume of the gas bubble included in the fluid delivered to the fluid line, a volume of the fluid delivered to the fluid line, or any combination thereof.

In some non-limiting embodiments or aspects, the pressure sensor includes at least one of the following: an absolute pressure sensor, a differential pressure sensor, or any combination thereof.

In some non-limiting embodiments or aspects, the microcontroller is further programmed and/or configured to: receive, from the pressure sensor, the pressure measured in the fluid pathway downstream of the pump during a priming of the pump; and determine, based on the pressure measured in the fluid pathway downstream of the pump during the priming of the pump, whether the pump is fully primed.

According to some non-limiting embodiments or aspects, provided is a method for pressure sensor based gas bubble detection for a drug delivery device comprising a fluid pathway including a reservoir configured to receive a fluid, a pump downstream of the reservoir, and a fluid line downstream of the pump, the method including: delivering, with the pump, the fluid from the reservoir to the fluid line; measuring, with a pressure sensor, a pressure in the fluid pathway downstream of the pump as the fluid is delivered to the fluid line; receiving, with a microcontroller, the pressure measured in the fluid pathway downstream of the pump as the fluid is delivered to the fluid line; determining, with the microcontroller, based on the pressure measured in the fluid pathway downstream of the pump as the fluid is delivered to the fluid line, whether the fluid delivered to the fluid line includes a gas bubble; and controlling, with the microcontroller, an output device to provide an indication associated with the determination of whether the fluid delivered to the fluid line includes a gas bubble.

In some non-limiting embodiments or aspects, the pump includes a dosing chamber, wherein the pump is configured to cyclically (i) pump the reservoir with the dosing chamber in fluid communication with the reservoir and not in fluid communication with the fluid line and (ii) fluidically connect the dosing chamber with the fluid line, and wherein the fluid is delivered to the fluid line when the dosing chamber is fluidically connected with the fluid line.

In some non-limiting embodiments or aspects, the method further includes: before fluidically connecting the dosing chamber with the fluid line: measuring, with the pressure sensor, a pressure in the fluid pathway downstream of the pump; and determining, based on the pressure measured in the fluid pathway downstream of the pump before fluidically connecting the dosing chamber with the fluid line, a baseline pressure, wherein whether the fluid delivered to the fluid line includes a gas bubble is determined based on the pressure measured in the fluid pathway downstream of the pump as the fluid is delivered to the fluid line and the baseline pressure.

In some non-limiting embodiments or aspects, determining whether the fluid delivered to the fluid line includes a gas bubble includes: comparing the pressure measured in the fluid pathway downstream of the pump as the fluid is delivered to the fluid line to a threshold pressure; and in response to the pressure measured in the fluid pathway downstream of the pump as the fluid is delivered to the fluid line satisfying the threshold pressure, determining that the fluid delivered to the fluid line includes a gas bubble.

In some non-limiting embodiments or aspects, determining whether the fluid delivered to the fluid line includes a gas bubble includes: determining, based on the pressure measured in the fluid pathway downstream of the pump as the fluid is delivered to the fluid line, a rate of change associated with the pressure measured in the fluid pathway downstream of the pump as the fluid is delivered to the fluid line; comparing the rate of change associated with the pressure measured in the fluid pathway downstream of the pump as the fluid is delivered to the fluid line to a threshold rate of change; and in response to the rate of change associated with the pressure measured in the fluid pathway downstream of the pump as the fluid is delivered to the fluid line satisfying the threshold rate of change, determine that the fluid delivered to the fluid line includes a gas bubble.

In some non-limiting embodiments or aspects, the method further includes: receiving, with the microcontroller, a pressure associated with the fluid pathway upstream of the pump; determining, with the microcontroller, based on the pressure associated with the fluid pathway upstream of the pump, the pressure measured in the fluid pathway downstream of the pump as the fluid is delivered to the fluid line, and dimensions of the fluid pathway, at least one of a volume of the gas bubble included in the fluid delivered to the fluid line, a volume of the fluid delivered to the fluid line, or any combination thereof.

In some non-limiting embodiments or aspects, determining whether the fluid delivered to the fluid line includes a gas bubble includes: determining, based on the pressure measured in the fluid pathway downstream of the pump as the fluid is delivered to the fluid line, a variation in the pressure over a period of time; comparing the variation in the pressure over the period of time to a threshold variation; and in response to the variation in the pressure over the period of time satisfying the threshold variation, determining that the fluid delivered to the fluid line during the period of time includes a gas bubble.

In some non-limiting embodiments or aspects, the method further includes: determining, with the microcontroller, based on the variation in the pressure over the period of time, at least one of a volume of the gas bubble included in the fluid delivered to the fluid line, a volume of the fluid delivered to the fluid line, or any combination thereof.

In some non-limiting embodiments or aspects, the method further includes: measuring, with the pressure sensor, another pressure in the fluid pathway downstream of the pump during a priming of the pump; receiving, with the microcontroller, from the pressure sensor, the another pressure measured in the fluid pathway downstream of the pump during the priming of the pump; and determining, based on the pressure measured in the fluid pathway downstream of the pump during the priming of the pump, whether the pump is fully primed.

According to some non-limiting embodiments or aspects, provided is a computer program product for pressure sensor based gas bubble detection for a drug delivery device including a microcontroller, a pressure sensor, and a fluid pathway including a reservoir configured to receive a fluid, a pump downstream of the reservoir, and a fluid line downstream of the pump, the computer program product comprising at least one non-transitory computer-readable medium including program instructions that, when executed by the microcontroller, cause the microcontroller to: control the pump to deliver the fluid from the reservoir to the fluid line; control the pressure sensor to measure a pressure in the fluid pathway downstream of the pump as the fluid is delivered to the fluid line; and receive, from the pressure sensor, the pressure measured in the fluid pathway downstream of the pump as the fluid is delivered to the fluid line; determine, based on the pressure measured in the fluid pathway downstream of the pump as the fluid is delivered to the fluid line, whether the fluid delivered to the fluid line includes a gas bubble; and control an output device to provide an indication associated with the determination of whether the fluid delivered to the fluid line includes a gas bubble.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a drug delivery device according to one aspect or embodiment of the present application.

FIG. 2 is a perspective view of the drug delivery device of FIG. 1, with a top cover removed.

FIG. 3 is a schematic of the drug delivery device of FIG. 1.

FIG. 4 is a schematic of a system for pressure sensor based gas bubble detection according to one aspect or embodiment of the present application.

FIGS. 5A and 5B are graphs of example pressure signatures and motor current signature for the drug delivery device of FIG. 1.

FIG. 6 is a graph of example pressure signatures and motor current signatures for the drug delivery device of FIG. 1.

FIG. 7 is a graph of an example pressure signature for the drug delivery device of FIG. 1.

FIG. 8 is a graph of another example pressure signature for the drug delivery device of FIG. 1.

FIG. 9 is a graph of further example pressure signatures and motor current signatures for the drug delivery device of FIG. 1.

FIG. 10 is a flow chart of a process for pressure sensor based gas bubble detection for a drug delivery device according to one aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION

Spatial or directional terms, such as “left”, “right”, “inner”, “outer”, “above”, “below”, and the like, are not to be considered as limiting as aspects or embodiments of the present disclosure can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

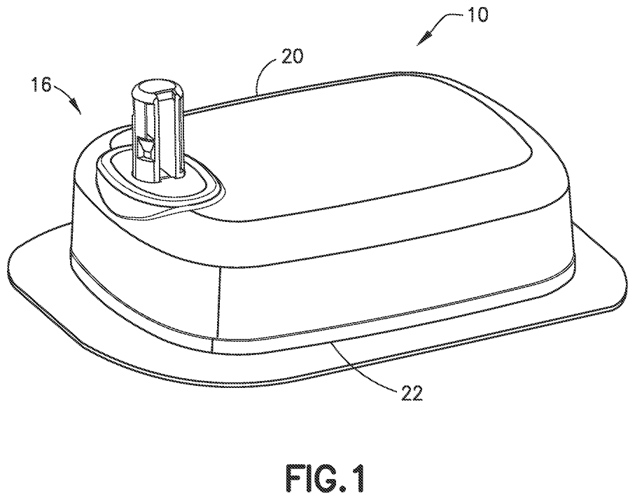

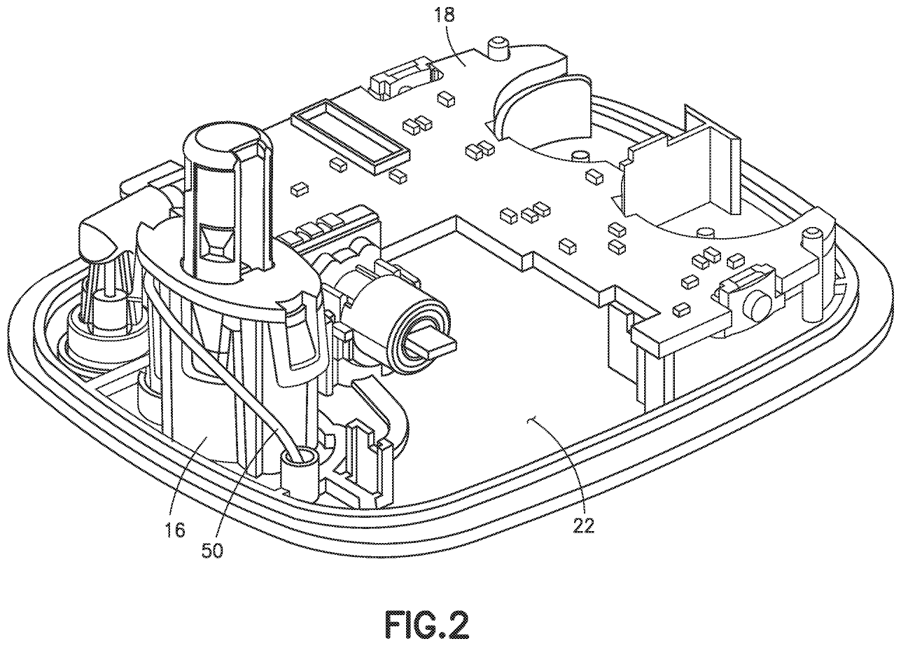

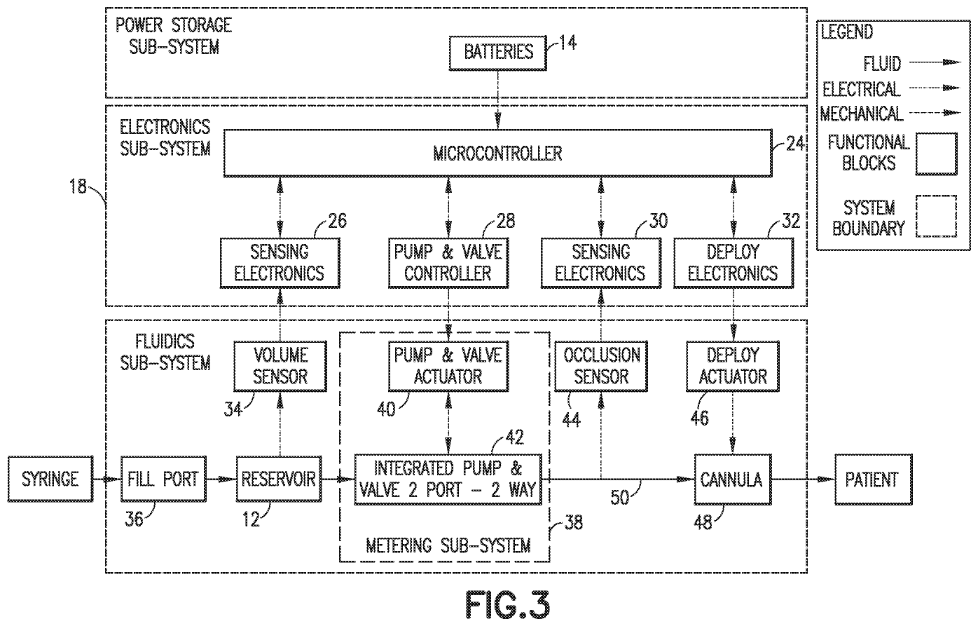

Referring to FIGS. 1-3, a drug delivery device 10 includes a reservoir 12, a power source 14, an insertion mechanism 16, control electronics 18, a cover 20, and a base 22. In one aspect or embodiment, the drug delivery device 10 is a wearable automatic injector, such as an insulin or bone marrow stimulant delivery device. The drug delivery device 10 may be mounted onto the skin of a patient and triggered to inject a pharmaceutical composition from the reservoir 12 into the patient. The drug delivery device 10 may be pre-filled with the pharmaceutical composition, or it may be filled with the pharmaceutical composition by the patient or medical professional prior to use.

The drug delivery device 10 is configured to deliver a dose of a pharmaceutical composition, e.g., any desired medicament, into the patient's body by a subcutaneous injection at a slow, controlled injection rate. Exemplary time durations for the delivery achieved by the drug delivery device 10 may range from about 5 minutes to about 60 minutes, but are not limited to this exemplary range. Exemplary volumes of the pharmaceutical composition delivered by the drug delivery device 10 may range from about 0.1 milliliters to about 10 milliliters, but are not limited to this exemplary range. The volume of the pharmaceutical composition delivered to the patient may be adjusted.

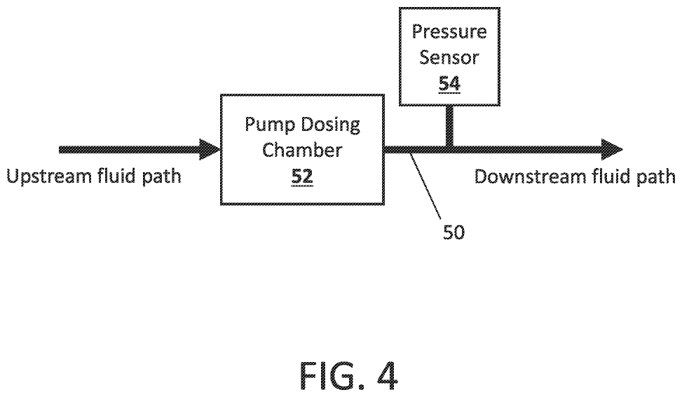

Referring again to FIGS. 1-3, in one aspect or embodiment, the power source 14 is a DC power source including one or more batteries. The control electronics 18 include a microcontroller 24, sensing electronics 26, a pump and valve controller 28, sensing electronics 30, and deployments electronics 32, which control the actuation of the drug delivery device 10. The drug delivery device 10 includes a fluidics sub-system that includes the reservoir 12, a volume sensor 34 for the reservoir 12, a reservoir fill port 36, and a metering system 38 including a pump and valve actuator 40 and a pump and valve mechanism 42. The fluidic sub-system may further include an occlusion sensor 44, a deploy actuator 46, a cannula 48 for insertion into a patient's skin, and a fluid line 50 in fluid communication with the reservoir 12 and the cannula 48. In one aspect or embodiment, occlusion sensor 44 includes a pressure sensor, such as pressure senor 54 described in more detail herein with respect to FIG. 4. In one aspect or embodiment, the insertion mechanism 16 is configured to move the cannula 48 from a retracted position positioned entirely within the device 10 to an extended position where the cannula 48 extends outside of the device 10. The drug delivery device 10 may operate in the same manner as discussed in U.S. Pat. No. 10,449,292 to Pizzochero et al, incorporated herein by reference.

In one aspect or embodiment, a fluid pathway is formed by the reservoir 12, the pump and valve mechanism 42 downstream of the reservoir 12, and the fluid line 50 downstream of the pump and valve mechanism 42. For example, the reservoir 12 may be configured to receive a fluid, and the pump and valve mechanism 42 may be configured to deliver the fluid from the reservoir 12 to the fluid line 50.

Referring to FIG. 4, in one aspect or embodiment, pump and valve mechanism 42 includes a dosing chamber 52. The pump and valve mechanism 42 may be configured to cyclically (i) pump the reservoir 12 with the dosing chamber 52 in fluid communication with the reservoir 12 and not in fluid communication with the fluid line 50 and (ii) fluidically connect the dosing chamber 52 with the fluid line 12 (e.g., with the dosing chamber 52 not in fluid communication with the reservoir 12, etc.). For example, the pump and valve mechanism 42 may pump the reservoir 12 with the dosing chamber 52 in fluid communication with the reservoir 12 and not in fluid communication with the fluid line 50 to fill the dosing chamber 52 with the fluid from the reservoir 12, and the pump and valve mechanism 42 may connect the dosing chamber 52 with the fluid line 50 to empty the dosing chamber 52 of the fluid and deliver the fluid to the fluid line 50 (e.g., with the dosing chamber 52 not in fluid communication with the reservoir 12, etc.). In this way, the fluidic path upstream of the pump and valve mechanism 42 (e.g., upstream of the dosing chamber 52, etc.) may not be in direct fluid communication with the fluidic path downstream of the pump and valve mechanism 42, allowing for different pressures to be established in these respective upstream and downstream fluid paths.

Still referring to FIG. 4, the drug delivery device 10 may include a pressure sensor 54. The pressure sensor 54 may be configured to measure a pressure in the fluid pathway formed by the by the reservoir 12, the pump and valve mechanism 42 downstream of the reservoir 12, and the fluid line 50 downstream of the pump and valve mechanism 42. The pressure sensor 54 may be in the fluid pathway downstream of the pump and valve mechanism 42 and/or in the fluid pathway upstream of the pump and valve mechanism 42. For example, the pressure sensor 54 may be configured to measure the pressure in the fluid pathway downstream of the pump and valve mechanism 42 (e.g., downstream of the dosing chamber 52, etc.) and/or in the fluid pathway upstream of pump and valve mechanism 42 (e.g., upstream of the dosing chamber 52, etc.). The pressure sensor 54 may include at least one of the following: an absolute pressure sensor, a differential (e.g., gauge, etc.) pressure sensor, or any combination thereof. The pressure sensor 54 may be miniaturized, may have high resolution, may be cost effective, and/or may be optimized for low power consumption.

The microcontroller 24 may be programmed and/or configured to: receive, from the pressure sensor 54, the pressure measured in the fluid pathway downstream of the pump and valve mechanism 42 as the fluid is delivered to the fluid line (e.g., with the dosing chamber 52 in fluid communication with the downstream fluid pathway including the fluid line 50, etc.); determine, based on the pressure measured in the fluid pathway downstream of the pump and valve mechanism 42 as the fluid is delivered to the fluid line 50 (e.g., the pressure measured with the dosing chamber 52 in fluid communication with the downstream fluid pathway including the fluid line 50, etc.), whether the fluid delivered to the fluid line includes a gas bubble(s); and/or control an output device (e.g., a display, a light emitting diode (LED), a speaker, etc.) to provide an indication associated with the determination of whether the fluid delivered to the fluid line includes a gas bubble(s). The output device may be included in and/or integrated with the drug delivery device 10 and/or the output device may be included in and/or integrated with an external device external to and in communication with (e.g., wireless and/or wireless communication, etc.) the drug delivery device 10, such as a remote computing device and/or a wireless controller (WC) 500 as discussed in U.S. Pat. No. 10,449,292 to Pizzochero et al, incorporated herein by reference.

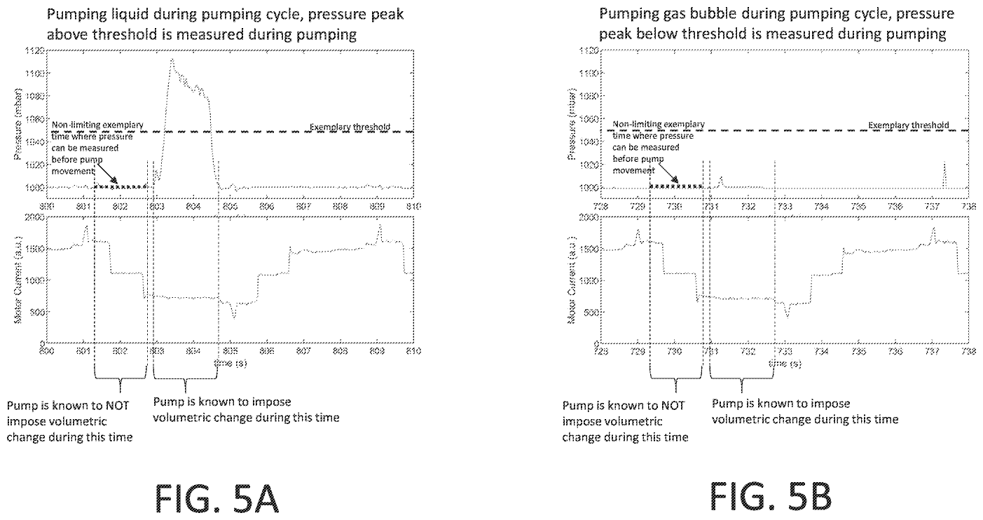

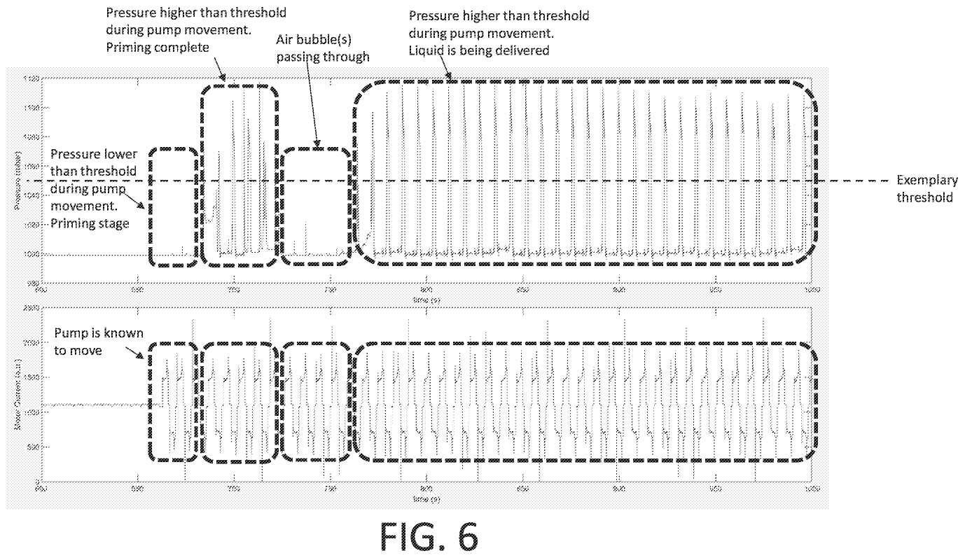

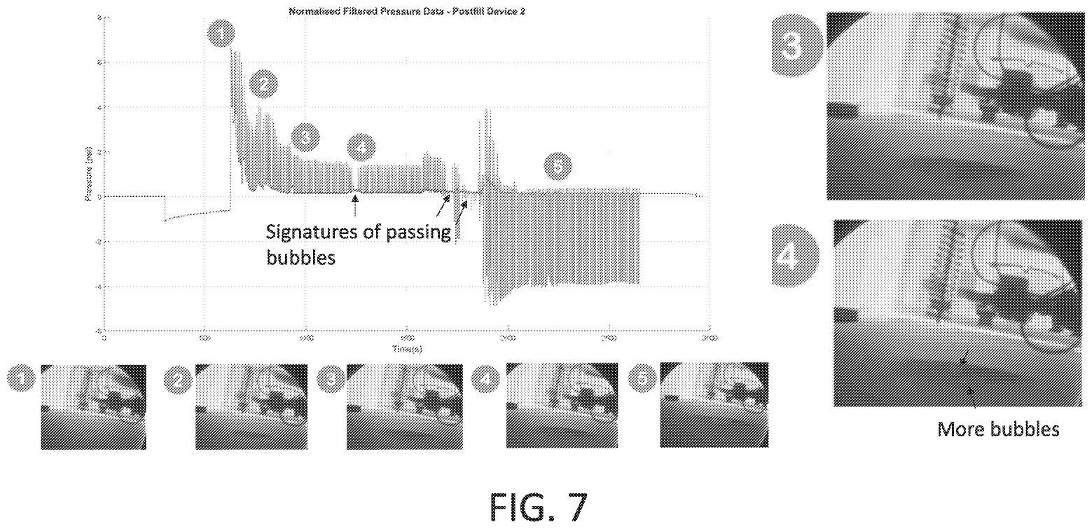

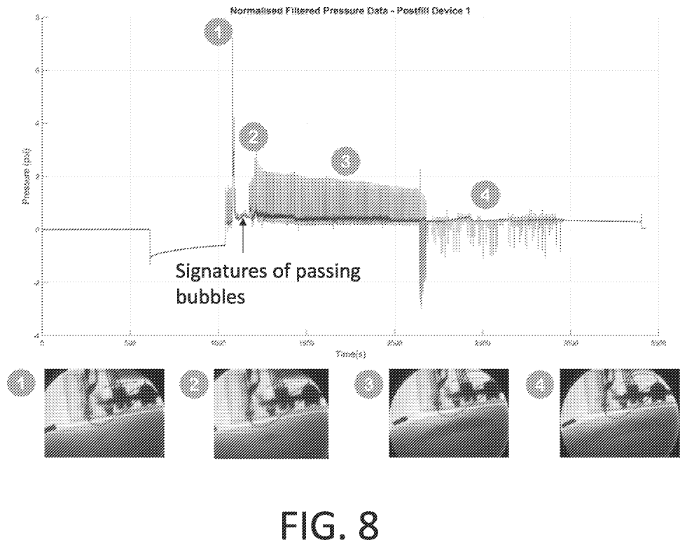

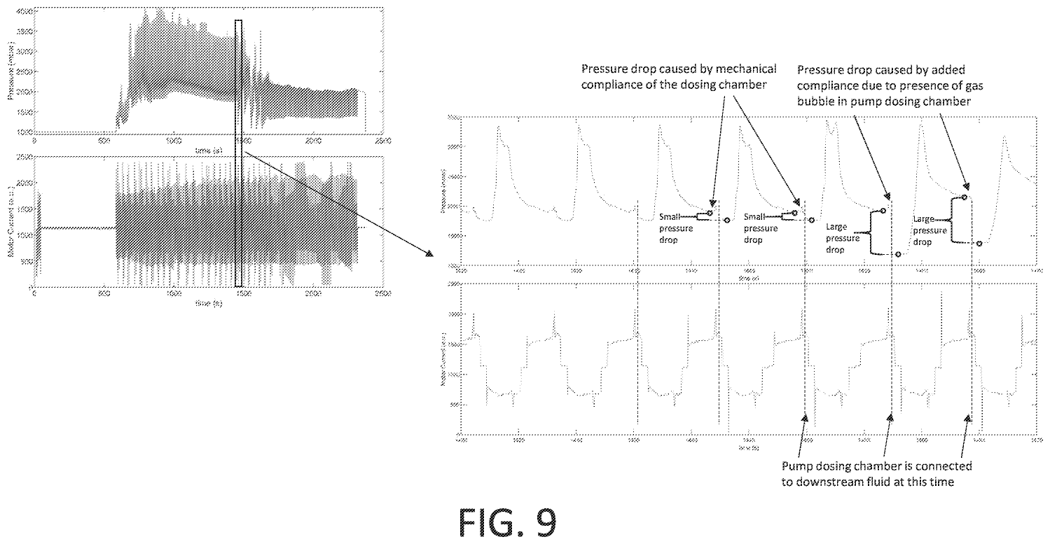

In this way, and referring also to FIGS. 5A, 5B and 6-9, non-limiting embodiments or aspects of the present application may exploit the higher compressibility of gases compared to liquids, combined with cyclical imposition of known volumetric change within the dosing chamber 52 and a pressure sensor positioned downstream relative to the pumping system with at least a portion of the fluid pathway connected to the fluid line 50 (e.g., to the injection port, etc.), to detect a pressure signature characteristic of a gas bubble(s) being pumped through the fluidic system. For example, when the pump and valve mechanism 42 imposes a known volumetric change within a known time period, a pressure in the downstream fluid pathway downstream of the dosing chamber 52 is expected to increase by at least a known value as a result of fluid movement in the fluid line 50. However, the presence of a gas bubble in the volume being pumped dampens the pressure increase and may be reliably detected by the pressure sensor 54. Moreover, the added compliance of a gas bubble in the dosing chamber 52 along with the pressure difference between the upstream and downstream fluid pathways relative to the dosing chamber 52 may cause a pressure change when the dosing chamber becomes in fluid communication with the downstream fluid line 50. If the pressure is higher in the downstream fluid pathway, a pressure drop may be identified as a signature or presence of an air or gas bubble(s). Using the pressure of the upstream fluid path, either by direct measurement or by inference (e.g. known to be at atmospheric pressure, etc.), a volume of the gas bubble(s) may be determined based on at least one of the pressure change in downstream fluid path, a volume of the fluidic system, dimensions of the fluidic system, the rheological properties of the fluid in the fluid line, or any combination thereof.

In one aspect or embodiment, the microcontroller 24 is programmed and/or configured to determine whether the fluid delivered to the fluid line 50 includes a gas bubble by: comparing the pressure measured in the fluid pathway downstream of the pump and valve mechanism 42 as the fluid is delivered to the fluid line 50 (e.g., a maximum or peak pressure achieved with the with the dosing chamber 52 in fluid communication with the downstream fluid pathway including the fluid line 50, a change or variation in the pressure in the downstream fluid pathway over a period of time in response to fluidically connecting the dosing chamber 52 with the downstream fluid pathway including the fluid line 50, etc.) to a threshold pressure (e.g., a threshold peak pressure, a threshold pressure change or variation, etc.); and in response to the pressure measured in the fluid pathway downstream of the pump and valve mechanism 42 as the fluid is delivered to the fluid line 50 satisfying the threshold pressure satisfying the threshold pressure, determining that the fluid delivered to the fluid line 50 includes a gas bubble. For example, the microcontroller 24 may compare a peak pressure reached during pumping (e.g., when the dosing chamber 52 of the pump and valve mechanism 42 is connected to the downstream fluid path, etc.) to the threshold pressure and, in response to the peak pressure satisfying the threshold pressure (e.g., in response to the peak pressure being below the threshold pressure, in response to a pressure or change or variation being above the threshold pressure change, etc.) determine that the that the fluid delivered to the fluid line 50 includes a gas bubble.

In one aspect or embodiment, the microcontroller 24 is programmed and/or configured to determine whether the fluid delivered to the fluid line includes a gas bubble by: determining, based on the pressure measured in the fluid pathway downstream of the pump and valve mechanism 42 as the fluid is delivered to the fluid line 50 (e.g., when the dosing chamber 52 of the pump and valve mechanism 42 is connected to the downstream fluid path, etc.), a rate of change associated with the pressure measured in the fluid pathway downstream of the pump as the fluid is delivered to the fluid line; comparing the rate of change associated with the pressure measured in the fluid pathway downstream of the pump and valve mechanism 42 as the fluid is delivered to the fluid line 50 to a threshold rate of change; and in response to the rate of change associated with the pressure measured in the fluid pathway downstream of the pump and valve mechanism 42 as the fluid is delivered to the fluid line 50 satisfying the threshold rate of change, determine that the fluid delivered to the fluid line 50 includes a gas bubble. For example, the microcontroller 24 may compare the rate of change associated with the pressure measured in the fluid pathway downstream of the pump and valve mechanism 42 during pumping (e.g., a rate of pressure increase, a rate of pressure decrease, a rate of pressure increase rate, a rate of pressure decrease rate, etc.) to the threshold rate of change and, in response to the measured rate of change satisfying the threshold rate (e.g., falling below the threshold rate, etc.), determine that the fluid delivered to the fluid line 50 includes a gas bubble.

In one aspect or embodiment, the microcontroller 24 is further programmed and/or configured to: receive, from the pressure sensor 54, a pressure measured in the fluid pathway downstream of the pump and valve mechanism 42 before fluidically connecting the dosing chamber 52 with the fluid line 50 (e.g., before initiating a volumetric change in the downstream fluid pathway, etc.); and determine, based on the pressure measured in the fluid pathway downstream of the pump and valve mechanism 42 before fluidically connecting the dosing chamber 52 with the fluid line 50, a baseline pressure. In such an example, the microcontroller 24 may be programmed and/or configured to determine whether the fluid delivered to the fluid line 50 includes a gas bubble based on the pressure measured in the fluid pathway downstream of the pump as the fluid is delivered to the fluid line 50 and the baseline pressure. For example, the measured pressure signature in the fluid pathway may be offset by a baseline pressure signature defined by a value of the pressure measured before connecting the dosing chamber 52 to the downstream fluid pathway. In this way, non-limiting embodiments or aspects of the present application may compensate for an influence on the pressure signature due to mechanical compliance of the pumping system (e.g. spring effect, deformability of components, mechanical backlash, etc.) when the dosing chamber 52 is connected to the downstream fluid pathway. For example, the pressure signature measured when the dosing chamber 52 is connected to the downstream fluid pathway may be exploited to detect the presence of air or gas in response to a pressure variation above a predefined threshold after compensation of system effect.

In one aspect or embodiment, the microcontroller 24 is further programmed and/or configured to: receive a pressure associated with the fluid pathway upstream of the pump and valve mechanism 42 (e.g., a direct measurement by pressure sensor 54 or another pressure sensor in the upstream fluid pathway, a known atmospheric pressure, etc.); and determine, based on the pressure associated with the fluid pathway upstream of the pump and valve mechanism 42, the pressure measured in the fluid pathway downstream of the pump and valve mechanism 42 as the fluid is delivered to the fluid line 50, and dimensions of the fluid pathway (e.g., dimensions of the reservoir, the dosing chamber 52, the fluid line 50, the upstream fluid pathway, and/or the downstream fluid pathway, etc.), at least one of a volume of the gas bubble included in the fluid delivered to the fluid line 50, a volume of the fluid delivered to the fluid line 50, or any combination thereof. In this way, the pressure variation may be used to determine gas volume and/or liquid volume delivered to the downstream fluid path.

In one aspect or embodiment, the microcontroller 24 is further programmed and/or configured to: determine a relative volume of gas in the fluid delivered to the fluid line 50 by applying, to the measured pressure signature in the fluid pathway, a combination of the Hagen-Poiseuille equation and known gas laws considering at least one of the fluid properties, the pumping regimen, and the dimensions of the fluid channel. For example, microcontroller 24 may determine, based on the variation in the pressure over the period of time, at least one of a volume of the gas bubble included in the fluid delivered to the fluid line 50, a volume of the fluid delivered to the fluid line 50, or any combination thereof.

In one aspect or embodiment, the microcontroller 24 is further programmed and/or configured to: receive, from the pressure sensor 54, the pressure measured in the fluid pathway downstream of the pump and valve mechanism 42 during a priming of the pump and valve mechanism 42; and determine, based on the pressure measured in the fluid pathway downstream of the pump and valve mechanism 42 during the priming of the pump and valve mechanism 42, whether the pump and valve mechanism 42 is fully primed. For example, the microcontroller 24 may compare the pressure measured in the fluid pathway downstream of the pump and valve mechanism 42 during the priming of the pump and valve mechanism 42 (e.g., a maximum or peak pressure achieved during priming, a change or variation in the pressure during priming, etc.) to a threshold priming pressure and, in response to the measured priming pressure satisfying the threshold priming pressure, determine that the downstream fluid path is fully primed.

In one aspect or embodiment, the indication associated with the determination of whether the fluid delivered to the fluid line 50 includes a gas bubble includes a notification of a successfully completed therapy (e.g., no gas bubbles, etc.), a notification of a failure mode and/or a gas bubble included in the fluid delivered to the fluid line 50, a notification of a volume of the fluid delivered to the fluid line, a notification of a volume of a gas bubble in the fluid delivered to the fluid line, a notification of a priming state of the device 10 (e.g., fully primed, not fully primed, etc.), or any combination thereof.

In one aspect or embodiment, the microcontroller 24 may, in response to detecting a gas bubble, adjust the cyclic pumping of the pump and valve mechanism 42 to compensate for the detected gas bubble (e.g., by adding an additional pump cycle, by adjusting a duration of a pump cycle of a stage of a pump cycle, etc.). For example, the microcontroller 24 may be further programmed and/or configured to compare the volume of the gas bubble and/or the fluid delivered to a threshold or prescribed volume and, in response to the he volume of the gas bubble and/or the fluid delivered satisfying threshold or prescribed volume, automatically adjust the cyclic pumping of the pump and valve mechanism 42 to compensate for the volume of the detected gas bubble (e.g., by adding an additional pump cycle, by adjusting a duration of a pump cycle of a stage of a pump cycle, etc.).

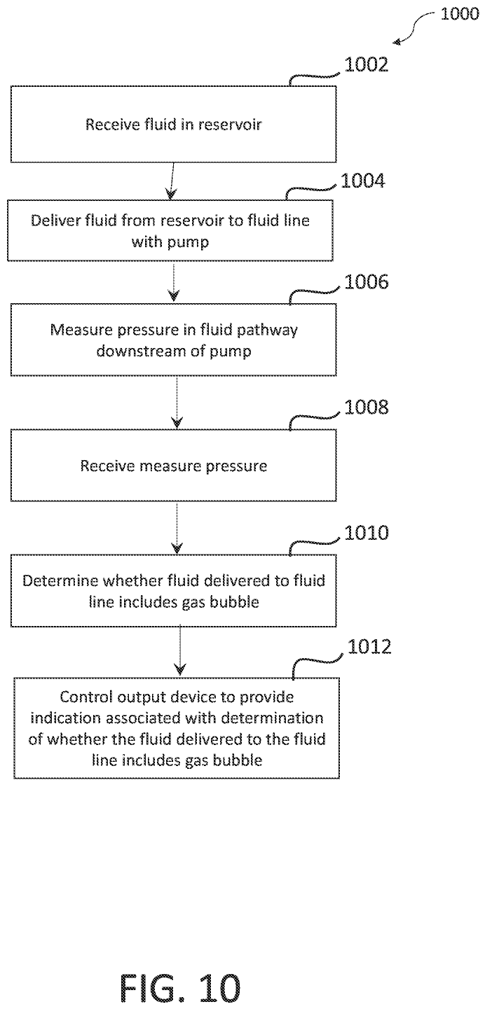

Referring to FIG. 10, in one aspect or embodiment, a process 1000 for pressure sensor based gas bubble detection for the drug delivery device 10 includes: receiving a fluid in the reservoir 12 (step 1002); delivering, with the pump and valve mechanism 42, the fluid from the reservoir 12 to the fluid line 50 (step 1004); measuring, with the pressure sensor 54, a pressure in the fluid pathway downstream of the pump and valve mechanism 42 as the fluid is delivered to the fluid line 50 (step 1006); receiving, with the microcontroller 24, the pressure measured in the fluid pathway (step 1008); determining, with the microcontroller 24, based on the pressure measured in the fluid pathway, whether the fluid delivered to the fluid line 50 includes a gas bubble (step 1010); and controlling, with the microcontroller 24, an output device to provide an indication associated with the determination of whether the fluid delivered to the fluid line 50 includes a gas bubble (step 1012).

Accordingly, non-limiting embodiments or aspects of the present application may provide a drug delivery device having a low cost of manufacture, extremely low power consumption, a compact size, and/or low computing power requirements that may leverage potentially existing hardware in a pumping system and/or may be used with a flexible reservoir bag, rigid containers, and/or syringe-like containers.

Although aspects or embodiments have been described in detail for the purpose of illustration based on what is currently considered to be the most practical and preferred embodiments, it is to be understood that such detail is solely for that purpose and that aspects or embodiments of the present disclosure are not limited to the disclosed embodiments, but, on the contrary, are intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A drug delivery device, comprising:
a fluid pathway including a reservoir, a pump downstream of the reservoir, and a fluid line downstream of the pump, wherein the reservoir is configured to receive a fluid, and wherein the pump is configured to deliver the fluid from the reservoir to the fluid line;
a pressure sensor configured to measure a pressure in the fluid pathway downstream of the pump; and
a microcontroller programmed and/or configured to:
receive, from the pressure sensor, the pressure measured in the fluid pathway downstream of the pump as the fluid is delivered to the fluid line;
determine, based on the pressure measured in the fluid pathway downstream of the pump as the fluid is delivered to the fluid line, whether the fluid delivered to the fluid line includes a gas bubble; and
control an output device to provide an indication associated with the determination of whether the fluid delivered to the fluid line includes a gas bubble.

2. The drug delivery device of claim 1, wherein the pump includes a dosing chamber, wherein the pump is configured to cyclically (i) pump the reservoir with the dosing chamber in fluid communication with the reservoir and not in fluid communication with the fluid line and (ii) fluidically connect the dosing chamber with the fluid line, and wherein the fluid is delivered to the fluid line when the dosing chamber is fluidically connected with the fluid line.

3. The drug delivery device of claim 2, wherein the microcontroller is further programmed and/or configured to:
receive, from the pressure sensor, a pressure measured in the fluid pathway downstream of the pump before fluidically connecting the dosing chamber with the fluid line; and
determine, based on the pressure measured in the fluid pathway downstream of the pump before fluidically connecting the dosing chamber with the fluid line, a baseline pressure, wherein the microcontroller is programmed and/or configured to determine whether the fluid delivered to the fluid line includes a gas bubble based on the pressure measured in the fluid pathway downstream of the pump as the fluid is delivered to the fluid line and the baseline pressure.

4. The drug delivery device of claim 1, wherein the microcontroller is programmed and/or configured to determine whether the fluid delivered to the fluid line includes a gas bubble by:
comparing the pressure measured in the fluid pathway downstream of the pump as the fluid is delivered to the fluid line to a threshold pressure; and
in response to the pressure measured in the fluid pathway downstream of the pump as the fluid is delivered to the fluid line satisfying the threshold pressure, determining that the fluid delivered to the fluid line includes a gas bubble.

5. The drug delivery device of claim 1, wherein the microcontroller is programmed and/or configured to determine whether the fluid delivered to the fluid line includes a gas bubble by:
determining, based on the pressure measured in the fluid pathway downstream of the pump as the fluid is delivered to the fluid line, a rate of change associated with the pressure measured in the fluid pathway downstream of the pump as the fluid is delivered to the fluid line;
comparing the rate of change associated with the pressure measured in the fluid pathway downstream of the pump as the fluid is delivered to the fluid line to a threshold rate of change; and
in response to the rate of change associated with the pressure measured in the fluid pathway downstream of the pump as the fluid is delivered to the fluid line satisfying the threshold rate of change, determine that the fluid delivered to the fluid line includes a gas bubble.

6. The drug delivery device of claim 1, wherein the microcontroller is further programmed and/or configured to:
receive a pressure associated with the fluid pathway upstream of the pump; and
determine, based on the pressure associated with the fluid pathway upstream of the pump, the pressure measured in the fluid pathway downstream of the pump as the fluid is delivered to the fluid line, and dimensions of the fluid pathway, at least one of a volume of the gas bubble included in the fluid delivered to the fluid line, a volume of the fluid delivered to the fluid line, or any combination thereof.

7. The drug delivery device of claim 1, wherein the microcontroller is programmed and/or configured to determine whether the fluid delivered to the fluid line includes a gas bubble by:
determining, based on the pressure measured in the fluid pathway downstream of the pump as the fluid is delivered to the fluid line, a variation in the pressure over a period of time;
comparing the variation in the pressure over the period of time to a threshold variation; and
in response to the variation in the pressure over the period of time satisfying the threshold variation, determining that the fluid delivered to the fluid line during the period of time includes a gas bubble.

8. The drug delivery device of claim 7, wherein the microcontroller is further programmed and/or configured to:
determine, based on the variation in the pressure over the period of time, at least one of a volume of the gas bubble included in the fluid delivered to the fluid line, a volume of the fluid delivered to the fluid line, or any combination thereof.

9. The drug delivery device of claim 1, wherein the pressure sensor includes at least one of the following: an absolute pressure sensor, a differential pressure sensor, or any combination thereof.

10. The drug delivery device of claim 1, wherein the microcontroller is further programmed and/or configured to:
receive, from the pressure sensor, the pressure measured in the fluid pathway downstream of the pump during a priming of the pump; and
determine, based on the pressure measured in the fluid pathway downstream of the pump during the priming of the pump, whether the pump is fully primed.

11. A method for pressure sensor based gas bubble detection for a drug delivery device comprising a fluid pathway including a reservoir configured to receive a fluid, a pump downstream of the reservoir, and a fluid line downstream of the pump, the method comprising:
delivering, with the pump, the fluid from the reservoir to the fluid line;
measuring, with a pressure sensor, a pressure in the fluid pathway downstream of the pump as the fluid is delivered to the fluid line;
receiving, with a microcontroller, the pressure measured in the fluid pathway downstream of the pump as the fluid is delivered to the fluid line;
determining, with the microcontroller, based on the pressure measured in the fluid pathway downstream of the pump as the fluid is delivered to the fluid line, whether the fluid delivered to the fluid line includes a gas bubble; and
controlling, with the microcontroller, an output device to provide an indication associated with the determination of whether the fluid delivered to the fluid line includes a gas bubble.

12. The method of claim 11, wherein the pump includes a dosing chamber, wherein the pump is configured to cyclically (i) pump the reservoir with the dosing chamber in fluid communication with the reservoir and not in fluid communication with the fluid line and (ii) fluidically connect the dosing chamber with the fluid line, and wherein the fluid is delivered to the fluid line when the dosing chamber is fluidically connected with the fluid line.

13. The method of claim 12, further comprising:
before fluidically connecting the dosing chamber with the fluid line:
measuring, with the pressure sensor, a pressure in the fluid pathway downstream of the pump; and
determining, based on the pressure measured in the fluid pathway downstream of the pump before fluidically connecting the dosing chamber with the fluid line, a baseline pressure, wherein whether the fluid delivered to the fluid line includes a gas bubble is determined based on the pressure measured in the fluid pathway downstream of the pump as the fluid is delivered to the fluid line and the baseline pressure.

14. The method of claim 11, wherein determining whether the fluid delivered to the fluid line includes a gas bubble includes:
comparing the pressure measured in the fluid pathway downstream of the pump as the fluid is delivered to the fluid line to a threshold pressure; and
in response to the pressure measured in the fluid pathway downstream of the pump as the fluid is delivered to the fluid line satisfying the threshold pressure, determining that the fluid delivered to the fluid line includes a gas bubble.

15. The method of claim 11, wherein determining whether the fluid delivered to the fluid line includes a gas bubble includes:
determining, based on the pressure measured in the fluid pathway downstream of the pump as the fluid is delivered to the fluid line, a rate of change associated with the pressure measured in the fluid pathway downstream of the pump as the fluid is delivered to the fluid line;
comparing the rate of change associated with the pressure measured in the fluid pathway downstream of the pump as the fluid is delivered to the fluid line to a threshold rate of change; and
in response to the rate of change associated with the pressure measured in the fluid pathway downstream of the pump as the fluid is delivered to the fluid line satisfying the threshold rate of change, determine that the fluid delivered to the fluid line includes a gas bubble.

16. The method of claim 11, further comprising:
receiving, with the microcontroller, a pressure associated with the fluid pathway upstream of the pump;
determining, with the microcontroller, based on the pressure associated with the fluid pathway upstream of the pump, the pressure measured in the fluid pathway downstream of the pump as the fluid is delivered to the fluid line, and dimensions of the fluid pathway, at least one of a volume of the gas bubble included in the fluid delivered to the fluid line, a volume of the fluid delivered to the fluid line, or any combination thereof.

17. The method of claim 11, wherein determining whether the fluid delivered to the fluid line includes a gas bubble includes:
determining, based on the pressure measured in the fluid pathway downstream of the pump as the fluid is delivered to the fluid line, a variation in the pressure over a period of time;
comparing the variation in the pressure over the period of time to a threshold variation; and
in response to the variation in the pressure over the period of time satisfying the threshold variation, determining that the fluid delivered to the fluid line during the period of time includes a gas bubble.

18. The method of claim 17, further comprising:
determining, with the microcontroller, based on the variation in the pressure over the period of time, at least one of a volume of the gas bubble included in the fluid delivered to the fluid line, a volume of the fluid delivered to the fluid line, or any combination thereof.

19. The method of claim 11, further comprising:
measuring, with the pressure sensor, another pressure in the fluid pathway downstream of the pump during a priming of the pump;
receiving, with the microcontroller, from the pressure sensor, the another pressure measured in the fluid pathway downstream of the pump during the priming of the pump; and
determining, based on the pressure measured in the fluid pathway downstream of the pump during the priming of the pump, whether the pump is fully primed.

20. A computer program product for pressure sensor based gas bubble detection for a drug delivery device comprising a microcontroller, a pressure sensor, and a fluid pathway including a reservoir configured to receive a fluid, a pump downstream of the reservoir, and a fluid line downstream of the pump, the computer program product comprising at least one non-transitory computer-readable medium including program instructions that, when executed by the microcontroller, cause the microcontroller to:
control the pump to deliver the fluid from the reservoir to the fluid line;
control the pressure sensor to measure a pressure in the fluid pathway downstream of the pump as the fluid is delivered to the fluid line; and
receive, from the pressure sensor, the pressure measured in the fluid pathway downstream of the pump as the fluid is delivered to the fluid line;
determine, based on the pressure measured in the fluid pathway downstream of the pump as the fluid is delivered to the fluid line, whether the fluid delivered to the fluid line includes a gas bubble; and
control an output device to provide an indication associated with the determination of whether the fluid delivered to the fluid line includes a gas bubble.
